(19) United States
(12) Patent Application Publication
(10) Pub. No.: US 2007/0160981 A1
(43) Pub. Date: Jul. 12, 2007

(54) VIRAL PROTEASE

(76) Inventors: Xin Chen, Miaoli County (TW); Yu San Han, Hsinchu County (TW); Tsu-An Hsu, Taipei (TW)

Correspondence Address: FISH & RICHARDSON PC, P.O. BOX 1022, MINNEAPOLIS, MN, 55440-1022, US

(21) Appl. No.: 11/551,789

(22) Filed: Oct. 23, 2006

Related U.S. Application Data
(60) Provisional application No. 60/729,865, filed on Oct. 24, 2005.

(51) Int. Cl. C12Q 1/70; A61K 38/16; A61K 38/10; A61K 31/522

(52) U.S. Cl. 435/5; 514/12; 514/13; 514/14; 514/15; 530/328; 530/350; 514/263.33; 514/263.34; 435/456; 435/325; 514/263.37

(57) ABSTRACT

An isolated polypeptide comprising a sequence that includes X₋₅-X₋₄-X₋₃-X₋₂-X₋₁-X₊₁, wherein X₋₅ is Arg, Ala, Ser, or Glu; X₋₄ is Leu; X₋₃ is Lys, Ala, Gln, or Asn; X₋₂ is Gly or Ala; X₋₁ is Gly; and X₊₁ is Ala, Gly, Asn, Val, or Lys; and the polypeptide, upon contact with SARS-CoV PLP2, murine hepatitis virus PLP, or bovine coronavirus PLP, is cleaved between residues X₋₁ and X₊₁ Also disclosed are related nucleic acids, vectors, host cells, screening methods, and treatment methods.

### RELATED APPLICATIONS

This application claims priority to U.S. Application Ser. No. 60/729,865, filed on Oct. 24, 2005, the content of which is incorporated herein in its entirety.

### BACKGROUND

Virus is the cause of various disorders. For example, members of the coronavirus family cause hepatitis in mice, gastroenteritis in pigs, and respiratory infections in birds and humans. Among the more than 30 strains isolated so far, three or four infect humans. The severe acute respiratory syndrome (SARS), a newly found infectious disease, is associated with a novel coronavirus. This coronavirus touched off worldwide outbreaks in 2003 (Peiris et al., 2003, Lancet 361, 1767-1772; and Ksiazek et al., 2003, N. Engl. J. Med. 348, 1953-1966). Drugs against SARS coronavirus (SARS CoV) are being vigorously sought. Nevertheless, the progress has been rather slow due to safety concerns.

Viral proteases are essential for viral pathogenesis and virulence. Drugs targeting them can be used to treat SARS or other viral infection. Yet, little is known about the enzymatic properties and the substrate specificity of SARS-CoV proteases.

### SUMMARY

This invention is based, at least in part, on unexpected finding of a consensus substrate sequence of SARS-CoV papain-like protease 2 (SARS PLP2), murine hepatitis virus papain-like protease (MHV PLP), or bovine coronavirus papain-like protease (BCoV PLP).

Shown below are the polypeptide and nucleic acid sequences of SARS-CoV PLP2 (SEQ ID NOs: 1 and 2):

SEQ ID NO: 1:
LNSLNEPLVTMPIGYVTHGFNLEEAARCMRSLKAPAVVSVSSPDAVTTYN
GYLTSSSKTSEEHFVETVSLAGSYRDWSYSGQRTELGVEFLKRGDKIVYH
TLESPVEFHLDGEVLSLDKLKSLLSLREVKTIKVFTTVDNTNLHTQLVDM
SMTYGQQFGPTYLDGADVTKIKPHVNHEGKTFFVLPSDDTLRSEAFEYYH
TLDESFLGRYMSALNHTKKWKFPQVGGLTSIKWADNNCYLSSVLLALQQL
EVKFNAPALQEAYYRARAGDAANFCALILAYSNKTVGELGDVRETMTHLL
QHANLESAKRVLNVVCKHCGQKTTTLTGVEAVMYMGTLSYDNLKTGVSIP
CVCGRDATQYLVQQESSFVMMSAPPAEYKLQQGTFLCANEYTGNYQCGHY
THITAKETLYRIDGAHLTKMSEYKGPVTDVFYKETSYTTTIKPVS
SEQ ID NO: 2:
CTG AAC TCT CTA AAT GAG CCG CTT GTC ACA ATG CCA
ATT GGT TAT GTG ACA CAT GGT TTT AAT CTT GAA GAG
GCT GCG CGC TGT ATG CGT TCT CTT AAA GCT CCT GCC
GTA GTG TCA GTA TCA TCA CCA GAT GCT GTT ACT ACA
TAT AAT GGA TAC CTC ACT TCG TCA TCA AAG ACA TCT
GAG GAG CAC TTT GTA GAA ACA GTT TCT TTG GCT GGC
TCT TAC AGA GAT TGG TCC TAT TCA GGA CAG CGT ACA
GAG TTA GGT GTT GAA TTT CTT AAG CGT GGT GAC AAA
ATT GTG TAC CAC ACT CTG GAG AGC CCC GTC GAG TTT
CAT CTT GAC GGT GAG GTT CTT TCA CTT GAC AAA CTA
AAG AGT CTC TTA TCC CTG CGG GAG GTT AAG ACT ATA
AAA GTG TTC ACA ACT GTG GAC AAC ACT AAT CTC CAC
ACA CAG CTT GTG GAT ATG TCT ATG ACA TAT GGA CAG
CAG TTT GGT CCA ACA TAC TTG GAT GGT GCT GAT GTT
ACA AAA ATT AAA CCT CAT GTA AAT CAT GAG GGT AAG
ACT TTC TTT GTA CTA CCT AGT GAT GAC ACA CTA CGT
AGT GAA GCT TTC GAG TAC TAC CAT ACT CTT GAT GAG
AGT TTT CTT GGT AGG TAC ATG TCT GCT TTA AAC CAC
ACA AAG AAA TGG AAA TTT CCT CAA GTT GGT GGT TTA
ACT TCA ATT AAA TGG GCT GAT AAC AAT TGT TAT TTG
TCT AGT GTT TTA TTA GCA CTT CAA CAG CTT GAA GTC
AAA TTC AAT GCA CCA GCA CTT CAA GAG GCT TAT TAT
AGA GCC CGT GCT GGT GAT GCT GCT AAC TTT TGT GCA
CTC ATA CTC GCT TAC AGT AAT AAA ACT GTT GGC GAG
CTT GGT GAT GTC AGA GAA ACT ATG ACC CAT CTT CTA
CAG CAT GCT AAT TTG GAA TCT GCA AAG CGA GTT CTT
AAT GTG GTG TGT AAA CAT TGT GGT CAG AAA ACT ACT
ACC TTA ACG GGT GTA GAA GCT GTG ATG TAT ATG GGT
ACT CTA TCT TAT GAT AAT CTT AAG ACA GGT GTT TCC
ATT CCA TGT GTG TGT GGT CGT GAT GCT ACA CAA TAT
CTA GTA CAA CAA GAG TCT TCT TTT GTT ATG ATG TCT
GCA CCA CCT GCT GAG TAT AAA TTA CAG CAA GGT ACA
TTC TTA TGT GCG AAT GAG TAC ACT GGT AAC TAT CAG
TGT GGT CAT TAC ACT CAT ATA ACT GCT AAG GAG ACC
CTC TAT CGT ATT GAC GGA GCT CAC CTT ACA AAG ATG
TCA GAG TAC AAA GGA CCA GTG ACT GAT GTT TTC TAC
AAG GAA ACA TCT TAC ACT ACA ACC ATC AAG CCT GTG
TCG

Accordingly, one aspect of this invention features an isolated polypeptide containing a sequence that includes X₋₅-X₋₄-X₋₃-X₋₂-X₋₁-X₊₁, in which X₋₅ is Arg, Ala, Ser, or Glu; X₋₄ is Leu; X₋₃ is Lys, Ala, Gln, or Asn; X₋₂ is Gly or Ala; X₋₁ is Gly; and X₊₁ is Ala, Gly, Asn, Val, or Lys; and the polypeptide, upon contact with SARS-CoV PLP2, MHV PLP, or BCoV PLP, is cleaved between residues X₋₁ and X₊₁. That is, the protease cleaves the polypeptide between residues X−1 and X₊₁, as determined by the in vitro assays described in Examples 2-5 below or any analogous assays. Preferably, it cleaves at least 25% of the polypeptide after 12 hours of digestion.

In one embodiment, the sequence includes X₋₆-X₋₅-X₋₄-X₋₃-X₋₂-X₋₁-X₊₁-X₊₂-X₊₃, in which X₋₆ is Arg, Phe, or Ile; X₊₂ is Pro, Val, or Ile; and X₊₃ is Ile, Phe, Val, or Thr. For example, the sequence can include anyone selected from the group consisting of SEQ ID NOs: 5-30 (listed below), e.g., R₋₆E₋₅L₋₄N₋₃G₋₂G₋₁A₊₁V₊₂T₊₃RYV (SEQ ID NO: 29), F₋₆R₋₅L₄K₋₃G₋₂G₋₁A₊₁P₊₂I₊₃KGV (SEQ ID NO: 8), or I₋₆S₋₅L₋₄K₋₃G₋₂G₋₁K₊₁I₊₂V₊₃STC (SEQ ID NO:28).

| | |
|---|---|
| SEQ ID NO. | Xₙ: −6−5−4−3−2−1+1+2+3 |

| | |
|---|---|
| SEQ ID NO.: 5 | N N V F R L K G G A P I K G V T F G |
| SEQ ID NO.: 6 | N V F R L K G G A P I K G V T F |
| SEQ ID NO.: 7 | V F R L K G G A P I K G V T |
| SEQ ID NO.: 8 | F R L K G G A P I K G V |
| SEQ ID NO.: 9 | R L K G G A P I K G |
| SEQ ID NO.: 10 | F R L K G G A P I K |
| SEQ ID NO.: 11 | F R L K G G A P I |
| SEQ ID NO.: 12 | F R L K G G A P I K G V |
| SEQ ID NO.: 13 | V F R L K G G G P I K G V T |
| SEQ ID NO.: 14 | V F R L K G G N P I K G V T |
| SEQ ID NO.: 15 | V F R L K G G V P I K G V T |
| SEQ ID NO.: 16 | V F R L K A G A P I K G V T |
| SEQ ID NO.: 17 | V F R L K G G A P I K G F T |
| SEQ ID NO.: 18 | V F R L K G G A P I K G S T |
| SEQ ID NO.: 19 | V V R L K G G A P I K G V T |
| SEQ ID NO.: 20 | V A R L K G G A P I K G V T |
| SEQ ID NO.: 21 | V F R L K G G A P I K G V T |
| SEQ ID NO.: 22 | V F A L K G G A P I K G V T |
| SEQ ID NO.: 23 | V F S L K G G A P I K G V T |
| SEQ ID NO.: 24 | V F R L A G G A P I K G V T |
| SEQ ID NO.: 25 | V F R L Q G G A P I K G V T |
| SEQ ID NO.: 26 | P F S L K G G A V F S Y F V |
| SEQ ID NO.: 27 | P F S L K G G A V F S R M V |
| SEQ ID NO.: 28 | I S L K G G K I V S T C |
| SEQ ID NO.: 29 | R E L N G G A V T R Y V |
| SEQ ID NO.: 30 | F S L K G G A |

The polypeptide is at least 8 amino acids (aa.) in length, e.g., 2560 aa. or any integer number of aa. between 8 and 2560.

An “isolated polypeptide” refers to a polypeptide substantially free from naturally associated molecules, i.e., it is at least 75% (i.e., any number between 75% and 100%, inclusive) pure by dry weight. Purity can be measured by any appropriate standard method, for example, by column chromatography, polyacrylamide gel electrophoresis, or HPLC analysis. An isolated polypeptide of the invention can be purified from a natural source, produced by recombinant DNA techniques, or by chemical methods.

The invention also features an isolated nucleic acid that contains a sequence encoding one of the above-mentioned polypeptides or a complement thereof. Examples of the nucleic acid includes sequences encoding SEQ ID NOs: 5-30. A nucleic acid refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA. An “isolated nucleic acid” is a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express the polypeptide of this invention. For this purpose, one can operatively linked the nucleic acid to suitable regulatory sequences to generate an expression vector.

A vector refers to a nucleic acid molecule capable of transporting another nucleic acid to which it has been linked. The vector can be capable of autonomous replication or integrate into a host DNA. Examples of the vector include a plasmid, cosmid, or viral vector. The vector of this invention includes a nucleic acid in a form suitable for expression of the nucleic acid in a host cell. Preferably the vector includes one or more regulatory sequences operatively linked to the nucleic acid sequence to be expressed. A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vector can be introduced into host cells to produce the polypeptide of this invention.

Also within the scope of this invention is a host cell that contains the above-described nucleic acid. Examples include E. coli cells, insect cells (e.g., using baculovirus expression vectors), yeast cells, or mammalian cells. See e.g., Goeddel, (1990) Gene Expression Technology: Methods in Enzymology 185, Academic Press, San Diego, Calif. To produce a polypeptide of this invention, one can culture a host cell in a medium under conditions permitting expression of the polypeptide encoded by a nucleic acid of this invention, and purify the polypeptide from the cultured cell or the medium of the cell. Alternatively, the nucleic acid of this invention can be transcribed and translated in vitro, for example, using T7 promoter regulatory sequences and T7 polymerase.

A polypeptide of this invention can be used in a method of identifying an inhibitor of SARS-CoV PLP2, MHV PLP, or BCoV PLP. The method includes (a) mixing, in a first sample, a test compound, a polypeptide of this invention, and an enzyme selected from the group consisting of SARS-CoV PLP2, MHV PLP, and BCoV PLP; and (b) detecting a first level of cleavage of the polypeptide by the enzyme. The test compound is determined to be an inhibitor of SARS-CoV PLP2, MHV PLP, or BCoV PLP if the first level is lower than a second level determined from a second sample in the same manner, except the second sample is free of the compound. The enzyme can contain SEQ ID NO: 1 or a functional equivalent thereof.

Various suitable methods, such as mass spectrometry, can be used to detect the level of cleavage. To enhance sensitivity and speed up the identification, methods based on fluorescence resonance energy transfer (FRET) can be used. For this purpose, the polypeptide is labeled at its amino-terminus and carboxyl-terminus respectively by a first fluorophore and a second fluorophore. One of the first and second fluorophores is a donor fluorophore and the other is an acceptor fluorophore, so that, when the polypeptide is intact, the donor fluorophore and the acceptor fluorophore are in close proximity to allow fluorescence resonance energy transfer between them. To detect a level of cleavage of the polypeptide, one can monitor fluorescent emission change of either the acceptor fluorophore or the donor fluorophore upon irradiation of the donor fluorophore with an excitation light. The change is a function of the cleavage of the polypeptide. Any suitable donor-acceptor fluorophore pairs can be used. Exemplary donor fluorophore and acceptor fluorophore are o-aminobenzoyl (Abz) and N-ethylenediamine-2,4-dinitrophenyl amide (EDDNP), respectively, or EDANS and dabcyl, respectively.

The invention also features a method of identifying a compound for treating an infection with SARS-CoV, MHV, or BCoV. The method includes mixing a test compound and a polypeptide of this invention, and detecting presence of a binding between the test compound and the polypeptide. The test compound is determined to be a candidate compound for treating the infection if a binding exists between the compound and the polypeptide.

The invention further features a method of treating an infection with SARS-CoV, MHV, or BCoV. The method includes administering to a subject in need thereof an effective amount of the above-described polypeptide or an inhibitor of SARS-CoV PLP2, MHV PLP, or BCoV PLP. The term “treating” is defined as administration of a composition to a subject with the purpose to cure, alleviate, relieve, remedy, prevent, or ameliorate a disorder, the symptom of the disorder, the disease state secondary to the disorder, or the predisposition toward the disorder. An “effective amount” is an amount of the composition that is capable of producing a medically desirable result, e.g., as described above, in a treated subject. The inhibitor can be selected from the group consisting of a zinc ion, a zinc salt, Azathioprine, Thiram, Carmustine, Thimerosal, N-ethylmaleimide, and Merbromin. The zinc salt can be N-ethyl-N-phenyldithiocarbamic acid Zn, Hydroxypridine-2-thione Zn, Dibenzyl dithiocarbamic Zn, or ZnCl₂. In one embodiment, the inhibitor is a compound of formula (I): 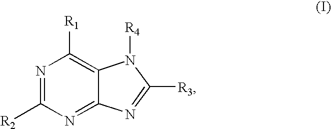
In the formula, (1) one of R₁, R₂, and R₃ is SR; and each of the others independently, is H, halo, C₁-C₁₅ alkyl, C₃-C₂₀ cycloalkyl, C₃-C₂₀ heterocycloalkyl, heteroaryl, aryl, SR, OR, or NRR′; and (2) R₄ is H, C₁-C₁₅ alkyl, C₃-C₂₀ cycloalkyl, C₃-C₂₀ heterocycloalkyl, heteroaryl, or aryl; in which each R and R′, independently, is H, C₁-C₁₅ alkyl, C₃-C₂₀ cycloalkyl, C₃-C₂₀ heterocycloalkyl, heteroaryl, or aryl; or its salt. Two examples of the compound are amino-6-mercaptopurine and 6-mercaptopurine.

The term “alkyl” refers to a saturated or unsaturated, linear or branched hydrocarbon moiety, such as —CH₃, —CH₂—CH═CH₂, or branched —C₃H₇. The term “cycloalkyl” refers to a saturated or unsaturated, non-aromatic, cyclic hydrocarbon moiety, such as cyclohexyl or cyclohexen-3-yl. The term “heterocycloalkyl” refers to a saturated or unsaturated, non-aromatic, cyclic moiety having at least one ring heteroatom (e.g., N, O, or S), such as 4-tetrahydropyranyl or 4-pyranyl. The term “aryl” refers to a hydrocarbon moiety having one or more aromatic rings. Examples of aryl moieties include phenyl (Ph), phenylene, naphthyl, naphthylene, pyrenyl, anthryl, and phenanthryl. The term “heteroaryl” refers to a moiety having one or more aromatic rings that contain at least one heteroatom (e.g., N, O, or S). Examples of heteroaryl moieties include furyl, furylene, fluorenyl, pyrrolyl, thienyl, oxazolyl, imidazolyl, thiazolyl, pyridyl, pyrimidinyl, quinazolinyl, quinolyl, isoquinolyl and indolyl.

Alkyl, cycloalkyl, heterocycloalkyl, aryl, and heteroaryl mentioned herein include both substituted and unsubstituted moieties, unless specified otherwise. Possible substituents on cycloalkyl, heterocycloalkyl, aryl, and heteroaryl include, but are not limited to, C₁-C₁₀ alkyl, C₃-C₈ cycloalkyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, hydroxyl, halogen, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, acyl, thioacyl, acyloxy, carboxyl, and carboxylic ester. On the other hand, possible substituents on alkyl include all of the above-recited substituents except C₁-C₁₀ alkyl. Cycloalkyl, heterocycloalkyl, aryl, and heteroaryl can also be fused with each other.

The details of one or more embodiments of the invention are set forth in the accompanying description below. Other advantages, features, and objects of the invention will be apparent from the detailed description, drawing, and the claims.

### DESCRIPTION OF DRAWING

FIG. 1 is a diagram showing predicted cleavage sites by SARS-CoV PLP2 (dark triangles) in non-structural polypeptide (nsp). The numbers at the top represent amino acid residues in nsp.

### DETAILED DESCRIPTION

This invention relates to polypeptides containing sites that are recognized and cleaved by SARS-CoV PLP2.

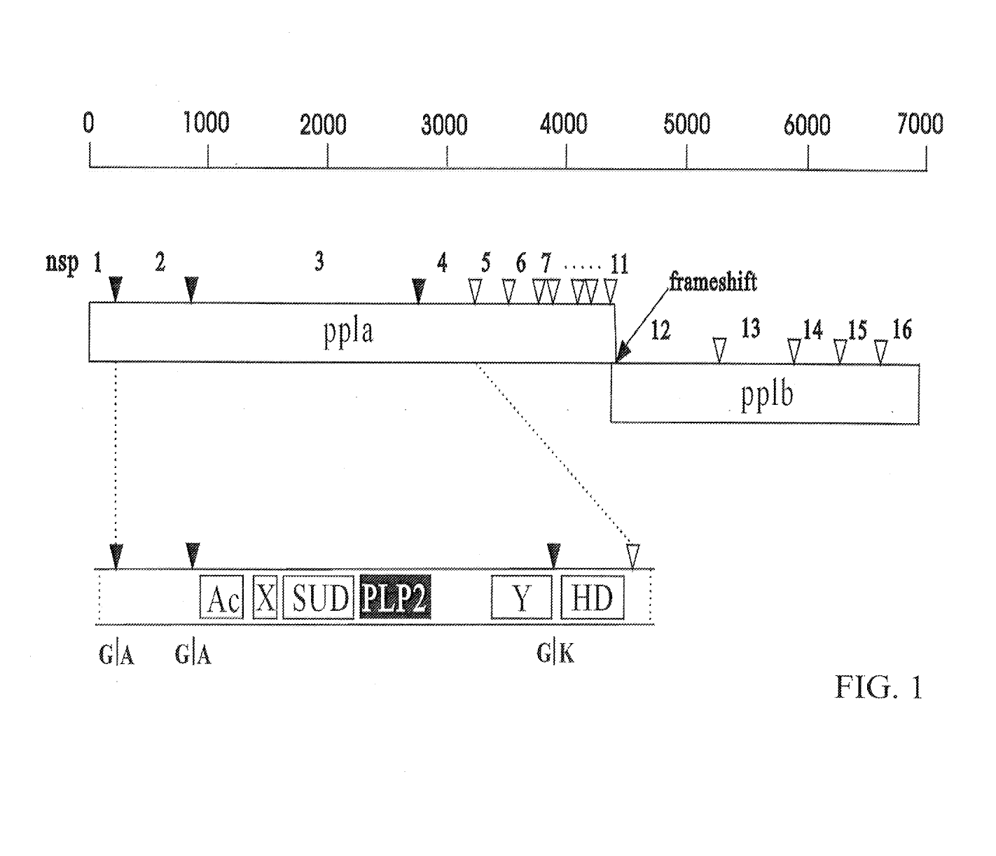

SARS-CoV (order Nidovirales, family Coronaviridae, genus Coronavirus) are enveloped positive-stranded RNA viruses (Marra et al., 2003, Science 300, 1399-1404; Rota et al., 2003, Science 300, 1394-1399; and Ruan et al., 2003, Lancet 361, 1779-1785). Its RNA, 29.7 kb in length, encodes structural proteins and two large nonstructural polypeptides, pp1a (486 kDa) and pp1ab (790 kDa). See FIG. 1. These two nonstructural polypeptides undergo co-translational processing that results in various non-structural proteins (nsps 1-16). It is believed that the processing of pp1a and pp1ab is performed by two viral proteases, 3C-like protease (3CL) and papain-like protease 2 (PLP2), included in pp1a (FIG. 1; Snijder et al., 2003, J. Mol. Biol. 331, 991-1004; Thiel et al., 2003, J. Gen. Virol. 84, 2305-2315; and Gao et al., 2003, FEBS Lett. 553, 451-456). The predicted sites to be cleaved by SARS-CoV PLP2 and SARS-CoV 3CL are shown in FIG. 1 as filled triangles and open triangles, respectively. Among nsps 1-16, nsp3, shown in the lower panel of FIG. 1, contains a number of domains, including the acidic, X, SARS-unique, papain-like protease 2, Y, and hydrophobic domains (domains Ac, X, SUD, PLP2, Y, and HD, respectively; Snijder et al., 2003, J. Mol. Biol. 331, 991-1004). Within this proteins are three cleavage sites at Gly180-Ala181 (G/A), Gly818-Ala819 (G/A) and Gly2740-Lys2741 (G/K).

A polypeptide of this invention contains a cleavage site of the SARS-CoV PLP2. Since the protease is essential for viral pathogenesis and virulence, the polypeptide can be targeted for treating an infection with the coronavirus. For example, the polypeptide can, via competition, inhibit the processing of the above-mentioned pp1a and pp1ab and thereby inhibit the viral infection. By the same token, a compound that binds to the site, by masking SARS-CoV PLP2 sites on pp1a and pp1ab, can also inhibit the infection. Further, the polypeptide can be used as a substrate for SARS-CoV PLP2 to screen inhibitors of SARS-CoV PLP2.

A polypeptide of the invention can be obtained as a synthetic polypeptide or a recombinant polypeptide. To prepare a recombinant polypeptide, a nucleic acid encoding it can be linked to another nucleic acid encoding a fusion partner, e.g., Glutathione-S-Transferase (GST), 6×-His epitope tag, or M13 Gene 3 protein. The resultant fusion nucleic acid expresses in suitable host cells a fusion protein that can be isolated by methods known in the art. The isolated fusion protein can be further treated, e.g., by enzymatic digestion, to remove the fusion partner and obtain the recombinant polypeptide of this invention.

A polypeptide of this invention can be used in a screening method of identifying an inhibitor of SARS-CoV PLP2, MHV PLP, or BCoV PLP. An inhibitor thus identified can be used for treating an infection with a coronavirus, e.g., SARS CoV, MHV, or BCoV. One screening method, as described in the Summary section above, includes (a) mixing, in a first sample, a test compound, a polypeptide of this invention, and an enzyme selected from the group consisting of SARS-CoV PLP2, MHV PLP, and BCoV PLP; and (b) detecting a first level of cleavage of the polypeptide by the enzyme. The test compound is determined to be an inhibitor of SARS-CoV PLP2, MHV PLP, or BCoV PLP if the first level is lower than a second level determined from a second sample in the same manner, except the second sample is free of the compound. The enzyme can be a protein containing SEQ ID NO: 1, its functional equivalent, or the equivalent sequence from the PLP2 protein of SARS CoV Urbani, TW1, Tor-2, SIN2500, SIN2774, SIN2748, SIN2677, SIN2679, CUHK-W1, HKU39849, GZ01, BJ01, BJ02, BJ03 BJ04, or other strains.

A functional equivalent of SEQ ID NO: 1 refers to a polypeptide derived from SEQ ID NO: 1, e.g., a fusion polypeptide or a polypeptide having one or more point mutations, insertions, deletions, truncations, or a combination thereof. In particular, such functional equivalents include polypeptides, whose sequences differ from SEQ ID NO: 1 by one or more conservative amino acid substitutions or by one or more non-conservative amino acid substitutions, deletions, or insertions. All of the just-mentioned functional equivalents have substantially the SARS-CoV PLP2 activity, i.e., to cleave a polypeptide of this invention between residues X−1 and X+1, as described in the Summary section above. This activity can be determined by the assays described in Examples 2-5 below or any analogous assays.

Within the scope of this invention is a method of identifying a compound for treating an infection with SARS-CoV, MHV, or BCoV. The method includes mixing a test compound and a polypeptide of this invention, and detecting presence of a binding between the test compound and the polypeptide. The test compound is determined to be a candidate compound for treating the infection if a binding exists between the compound and the polypeptide.

Test compounds (e.g., proteins, peptides, peptidomimetics, peptoids, antibodies, small molecules, or other drugs) can be obtained using any of the numerous approaches in combinatorial library methods known in the art. Such libraries include: peptide libraries, peptoid libraries (libraries of molecules having the functionalities of peptides, but with a novel, non-peptide backbone that is resistant to enzymatic degradation); spatially addressable parallel solid phase or solution phase libraries; synthetic libraries obtained by deconvolution or affinity chromatography selection; and the “one-bead one-compound” libraries. See, e.g., Zuckermann et al. 1994, J. Med. Chem. 37:2678-2685; and Lam, 1997, Anticancer Drug Des. 12:145. Examples of methods for the synthesis of molecular libraries can be found in, e.g., DeWitt et al., 1993, PNAS USA 90:6909; Erb et al., 1994, PNAS USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994 J. Med. Chem. 37:1233. Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (U.S. Pat. No. 5,223,409), spores (U.S. Pat. No. 5,223,409), plasmids (Cull et al., 1992, PNAS USA 89:1865-1869), or phages (Scott and Smith 1990, Science 249:386-390; Devlin, 1990, Science 249:404-406; Cwirla et al., 1990, PNAS USA 87:6378-6382; Felici 1991, J. Mol. Biol. 222:301-310; and U.S. Pat. No. 5,223,409).

A polypeptide of the invention can also be used to generate antibodies in animals (for production of antibodies) or humans (for treatment of diseases). Methods of making monoclonal and polyclonal antibodies and fragments thereof in animals are known in the art. See, for example, Harlow and Lane, (1988) Antibodies: A Laboratory Manual, Cold Spring Harbor Laboratory, New York. The term “antibody” includes intact molecules as well as fragments thereof, such as Fab, F(ab′)₂, Fv, scFv (single chain antibody), and dAb (domain antibody; Ward, et. al. (1989) Nature, 341, 544). These antibodies can be used for detecting the pp1a or pp1ab polypeptide, e.g., in determining whether a test sample (e.g., a tissue or cell lysate) from a subject contains coronavirus or in identifying a compound that binds to the polypeptide. As these antibodies interfere with a CoV PLP2's binding to and cleaving of its substrate they are also useful for treating a coronavirus infection.

In general, to produce antibodies against a polypeptide, the polypeptide is coupled to a carrier protein, such as KLH, mixed with an adjuvant, and injected into a host animal. Antibodies produced in the animal can then be purified by peptide affinity chromatography. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, CpG, surface-active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum.

Polyclonal antibodies, heterogeneous populations of antibody molecules, are present in the sera of the immunized subjects. Monoclonal antibodies, homogeneous populations of antibodies to a polypeptide of this invention, can be prepared using standard hybridoma technology (see, for example, Kohler et al. (1975) Nature 256, 495; Kohler et al. (1976) Eur. J. Immunol. 6, 511; Kohler et al. (1976) Eur J Immunol 6, 292; and Hammerling et al. (1981) Monoclonal Antibodies and T Cell Hybridomas, Elsevier, N.Y.). In particular, monoclonal antibodies can be obtained by any technique that provides for the production of antibody molecules by continuous cell lines in culture such as described in Kohler et al. (1975) Nature 256, 495 and U.S. Pat. No. 4,376,110; the human B-cell hybridoma technique (Kosbor et al. (1983) Immunol Today 4, 72; Cole et al. (1983) Proc. Natl. Acad. Sci. USA 80, 2026, and the EBV-hybridoma technique (Cole et al. (1983) Monoclonal Antibodies and Cancer Therapy, Alan R. Liss, Inc., pp. 77-96). Such antibodies can be of any immunoglobulin class including IgG, IgM, IgE, IgA, IgD, and any subclass thereof. The hybridoma producing the monoclonal antibodies of the invention may be cultivated in vitro or in vivo. The ability to produce high titers of monoclonal antibodies in vivo makes it a particularly useful method of production.

In addition, techniques developed for the production of “chimeric antibodies” can be used. See, e.g., Morrison et al. (1984) Proc. Natl. Acad. Sci. USA 81, 6851; Neuberger et al. (1984) Nature 312, 604; and Takeda et al. (1984) Nature 314:452. A chimeric antibody is a molecule in which different portions are derived from different animal species, such as those having a variable region derived from a murine monoclonal antibody and a human immunoglobulin constant region. Alternatively, techniques described for the production of single chain antibodies (U.S. Pat. Nos. 4,946,778 and 4,704,692) can be adapted to produce a phage library of single chain Fv antibodies. Single chain antibodies are formed by linking the heavy and light chain fragments of the Fv region via an amino acid bridge. Moreover, antibody fragments can be generated by known techniques. For example, such fragments include, but are not limited to, F(ab′)₂ fragments that can be produced by pepsin digestion of an antibody molecule, and Fab fragments that can be generated by reducing the disulfide bridges of F(ab′)₂ fragments. Antibodies can also be humanized by methods known in the art. For example, monoclonal antibodies with a desired binding specificity can be commercially humanized (Scotgene, Scotland; and Oxford Molecular, Palo Alto, Calif.). Fully human antibodies, such as those expressed in transgenic animals are also features of the invention (see, e.g., Green et al. (1994) Nature Genetics 7, 13; and U.S. Pat. Nos. 5,545,806 and 5,569,825).

A polypeptide of the invention (e.g., those containing SEQ ID NO.: 8, 28, or 29) can also be used to prepare an immunogenic composition (e.g., a vaccine) for generating antibodies against coronavirus (e.g., SRAS CoV) in a subject susceptible to the coronavirus. Such compositions can be prepared, e.g., according to the method described in the examples below, or by any other equivalent methods known in the art. The composition contains an effective amount of a polypeptide of the invention, and a pharmaceutically acceptable carrier such as phosphate buffered saline or a bicarbonate solution. The carrier is selected on the basis of the mode and route of administration, and standard pharmaceutical practice. Suitable pharmaceutical carriers and diluents, as well as pharmaceutical necessities for their use, are described in Remington's Pharmaceutical Sciences. An adjuvant, e.g., a cholera toxin, Escherichia coli heat-labile enterotoxin (LT), liposome, immune-stimulating complex (ISCOM), or immunostimulatory sequences oligodeoxynucleotides (ISS-ODN), can also be included in a composition of the invention, if necessary. A polypeptide of this invention may be components of a multivalent composition of vaccine against respiratory diseases. This multivalent composition contains at least one immunogenic fragment a polypeptide of this invention, along with at least one protective antigen isolated from influenza virus, para-influenza virus 3, Strentococcus pneumoniae, Branhamella (Moroxella) gatarhalis, Staphylococcus aureus, or respiratory syncytial virus, in the presence or absence of adjuvant.

Methods for preparing vaccines are generally well known in the art, as exemplified by U.S. Pat. Nos. 4,601,903; 4,599,231; 4,599,230; and 4,596,792. Vaccines may be prepared as injectables, as liquid solutions or emulsions. A polypeptide of this invention may be mixed with physiologically acceptable and excipients compatible. Excipients may include, water, saline, dextrose, glycerol, ethanol, and combinations thereof. The vaccine may further contain minor amounts of auxiliary substances such as wetting or emulsifying agents, pH buffering agents, or adjuvants to enhance the effectiveness of the vaccines. Methods of achieving adjuvant effect for the vaccine includes use of agents, such as aluminum hydroxide or phosphate (alum), commonly used as 0.05 to 0.1 percent solutions in phosphate buffered saline. Vaccines may be administered parenterally, by injection subcutaneously or intramuscularly. Alternatively, other modes of administration including suppositories and oral formulations may be desirable. For suppositories, binders and carriers may include, for example, polyalkalene glycols or triglycerides. Oral formulations may include normally employed incipients such as, for example, pharmaceutical grades of saccharine, cellulose, magnesium carbonate and the like. These compositions take the form of solutions, suspensions, tablets, pills, capsules, sustained release formulations or powders and contain 10-95% of the polypeptide of this invention.

The above-described polypeptides, inhibitors, and antibodies can be used for treating an infection with a coronavirus, e.g., SARS-CoV, MHV, or BCoV. Thus, within the scope of this invention is a method of treating such an infection, e.g., by administering to a subject in need thereof an effective amount of such a polypeptide, an antibody, or an inhibitor. Subjects to be treated can be identified as having, or being at risk for acquiring, a condition characterized by the infection. This method can be performed alone or in conjunction with other drugs or therapy.

Thus, also within the scope of this invention is a pharmaceutical composition that contains a pharmaceutically acceptable carrier and an effective amount of a polypeptide, an antibody, or an inhibitor of the invention. The pharmaceutical composition can be used to treat coronavirus infection. The pharmaceutically acceptable carrier includes a solvent, a dispersion medium, a coating, an antibacterial and antifungal agent, and an isotonic and absorption delaying agent.

In one in vivo approach, a composition of this invention is administered to a subject. Generally, polypeptide, the antibody, or the inhibitor is suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The dosage required depends on the choice of the route of administration; the nature of the formulation; the nature of the subject's illness; the subject's size, weight, surface area, age, and sex; other drugs being administered; and the judgment of the attending physician. Suitable dosages are in the range of 0.01-100.0 mg/kg. Wide variations in the needed dosage are to be expected in view of the variety of compositions available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the composition in a suitable delivery vehicle (e.g., polymeric microparticles or implantable devices) may increase the efficiency of delivery, particularly for oral delivery.

A pharmaceutical composition of the invention can be formulated into dosage forms for different administration routes utilizing conventional methods. For example, it can be formulated in a capsule, a gel seal, or a tablet for oral administration. Capsules can contain any standard pharmaceutically acceptable materials such as gelatin or cellulose. Tablets can be formulated in accordance with conventional procedures by compressing mixtures of the composition with a solid carrier and a lubricant. Examples of solid carriers include starch and sugar bentonite. The composition can also be administered in a form of a hard shell tablet or a capsule containing a binder, e.g., lactose or mannitol, conventional filler, and a tableting agent. The pharmaceutical composition can be administered via the parenteral route. Examples of parenteral dosage forms include aqueous solutions, isotonic saline or 5% glucose of the active agent, or other well-known pharmaceutically acceptable excipient. Cyclodextrins, or other solubilizing agents well known to those familiar with the art, can be utilized as pharmaceutical excipients for delivery of the therapeutic agent.

The efficacy of a composition of this invention can be evaluated both in vitro and in vivo. Briefly, the composition can be tested for its ability to inhibit the activity of a coronavirus PLP2 or the binding between the PLP2 and its substrate. For in vivo studies, the composition can be injected into an animal (e.g., a chimpanzee model) and its therapeutic effects are then accessed. Based on the results, an appropriate dosage range and administration route can be determined.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

Sequences of various coronavirus PLPs were studied. It was found that SARS-CoV, as well as infectious bronchitis virus (IBV), contains only one PLP domain, whereas both murine hepatitis virus (MHV) and human coronavirus 229E (HCoV-229E) have two paralogous PLP domains, PLP1 and PLP2. The classification of the PLP1 or PLP2 domain is based on its location in the non-structural protein. The PLP domains and others are usually organized in the order of acidic (Ac)-PLP1-X-PLP2-Y (Snijder et al., 2003, J. Mol. Biol. 331, 991-1004; and Ziebuhr et al., 2001, J. Biol. Chem. 276, 33220-33232). Because the PLP domains of both SARS-CoV and IBV are located between the X and Y domains, they are considered orthologues of PLP2.

Sequences of a number of coronaviral PLPs were aligned and shown below

| | |
|---|---|
| | 10 20 30 40 50 |
| PAPAIN | IPEYVDWRQKGAVTPVKNQGSCGSCWAFSAVVTIEGIIKIRTGNLNEYSEQELLDCDR-- |
| SARS_PLP2 | -----KWKFPQVGGLTSIKWADNNCYLSSVLLALQQLEVKFN---APALQEAYYRARAGD |
| BCoV_PLP2 | -----KWQVVFNGKYFTFKQANNNCFVNVSCLMLQSLNLKFK---IVQWQEAWLEFRSGR |
| MHV_PLP2 | -----KWPVVVCGNYFAFKQSNNNCYINVACLMLQHLSLKFH---KWQWQEAWNEFRSGK |
| IBV_PLP2 | -----KWNVQYRDNFLILEWRDGNCWISSAIVLLQAAKIRFKG----FLTEAWAKLLGGD |
| HCOV_229E_PLP2 | ------YESAVVNGIRVLKTSDNNCWVNAVCIALQYSKPHFIS---QGLDAAWNKFVLGD |
| HCOV_229E_PLP1 | -----EMPFEELNGLKILKQLDNNCWVNSVMLQIQLTGILDG-------DYAMQFFKMGR |
| BCoV_PLP1 | -----EPEFVKVLDLYVPKATRNNCWLRSVLAVMQKLPCQFK---DKNLQDLWVLYKQQY |
| MHV_PLP1 | ------ETHFKVCGFYSPAIERTNCWLRSTLIVMQSLPLEFK---DLEMQKLWLSYKSSY |
| | * |
| PAPAIN | RSYGCNGGYPWSALQLVAQYG-IHYRNTYPYEGVQRYCRSREKGPYA------------- |
| SARS_PLP2 | AANFCALILAYSN--KTVGELGDVRETMTHLLQHANLESAKRVLNVV--CKHCGQKTTTL |
| BCoV_PLP2 | PARFVSLVLAKGG-FKF-GDPADSRDFLRVVFSQVDLTGAICDFEIA--CK-CGVKQEQR |
| MHV_PLP2 | PLRFVSLVLAKGS-FKF-NEPSDSTDFMRVVLREADLSGATCDFEFV--CK-CGVKQEQR |
| IBV_PLP2 | PTDFVAWCYASCT-AKV-GDFSDANWLLANLAEHFDADYTNAFLKKRVSCN-CGIKSYEL |
| HCOV_229E_PLP2 | VEIFVAFVYYVARLMK--GDEGDAEDTLTKLSKYLANEAQVQLEHYS-SCVECDAKFKNS |
| HCOV_229E_PLP1 | VAKMIERCYTAE--QCIRGAMGDVGLCMYRLLKDLHTGFMVMDYK----CS-T---SGRL |
| BCoV_PLP1 | SQLFVDTLVNKIPANIVVPQGGYVADFAYWFLTLCDWQCVAYW-K----CIKCD-LALKL |
| MHV_PLP1 | NKEFVDLKVKSVPKSIILPQGGYVADFAYFFLSQCSFKAYANW-R----CLKCK-MDLKL |
| PAPAIN | ------------------------------AKTDGVRQVQPYNEGALLYSIANQ-PVSVV |
| SARS_PLP2 | TGVEAVMY-MGTLSYDNLKTGVSIPC-VCGRDATQYLVQQESSFVMMSAPP---AEYKLQ |
| BCoV_PLP2 | TGVDAVMH-FGTLSREDLEIGYTVDC-SCG-KKLIHCVRFDVPFLICSNTP---ASVKLP |
| MHV_PLP2 | KGVDAVMH-FGTLDKGDLAKGYTIAC-TCG-NKLVHCTQLNVPFLICSNKP---EGKKLP |
| IBV_PLP2 | RGLEACIQPVRATNLLHFKTQYSN-CPTCGANNTDEVIEASLPYLLLFATDG-PATVDCD |
| HCOV_229E_PLP2 | VASINSAI-VCASVKRDGVQVGY--C-VNGIKYYSRVRSVRGRAIIVSVEQLEPCAQSRL |
| HCOV_229E_PLP1 | EESGAVLF--CTPTKKAFPYGT---CLNCNAPRMCTIRQLQGTIIFVQQEPEPVNPVSFV |
| BCoV_PLP1 | KGLDAMFF------YGD-V--VSHVC-KCG-ESMVLIDVDVPFTAHFALKDKLFCAFITK |
| MHV_PLP1 | QGLDAMFF------YGD-V--VSHVC-KCG-TGMTLLSADIPYTLHFGLRDDKFCAFYTP |
| PAPAIN | LEAAGKDFQLYRGGIFVGPCGNKV--DHAVAAVGYGPNYILIKNS--------WGTGWGENGYIR |
| SARS_PLP2 | QGTFLCA----------NEYTGNYQCGHYTHITAKETLYRI--DGAHLTKMSEYEGPVTDVFYKE |
| BCoV_PLP2 | KG-VGSA----------NIFKG-DKVGHYVHVKCEQSYQLY--DASNVKKVTDVTGNLSDCLYLK |
| MHV_PLP2 | DD-V-VA---------ANIFTG-GSLGHYTHVKCKPKYQLY--DACNVSKVSEAKGNFTDCLYLK |
| IBV_PLP2 | EDAVGTV-----------VFVGSTNSGHCYTQAAGQAFDNLAKDRKFG-KKSPYITAMYTR---- |
| HCOV_229E_PLP2 | LSGVAYT-----------AFSGPVDEGHY-TVYDTAKKSMY--DGDRFVKHDLSLLSVTSVVMVG |
| HCOV_229E_PLP1 | VKPVCSS-----------IFRGAVSCGHYQTNIYSQNLCV---DGFGVNKIQPWTNDALNTICIK |
| BCoV_PLP1 | RS-VYKA-----------ACVVDVNDSHSMAVVDGKQI-----DDHRITSITSDKFDFIIGHGMS |
| MHV_PLP1 | RK-VFRA-----------ACVVDVNDCHSMAVVDGKQI-----DGKVVTKFNGDKYDFMVGHGMA |
| | * |

Based on sequence comparisons, it was predicted that all coronaviral PLPs, including SARS-CoV PLP2, are papain-like cysteine proteases with a catalytic dyad composed of Cys and His residues (“*”). The sequence homology of the coronaviral PLP domains is less than 25%, and the homology is even lower when compared with papain (Ziebuhr, J., Thiel, V., and Gorbalenya 2001, J. Biol. Chem. 276, 33220-33232). It is not known whether these coronaviral PLPs are similar to cellular papain structurally or catalytically.

A sequence alignment of PLP cleavage sites predicted that SARS-CoV PLP2 cleaves the first three sites on pp1a between Gly180 and Ala181, Gly818 and Ala819, and Gly2740 and Lys2741 of SARS-CoV (FIG. 1). However, limited information is available on the catalytic properties of coronaviral PLP proteases, possibly due to difficulties in expressing and purifying sufficient amounts of active PLP2 protein and lack of a sensitive and quantitative assay.

In this study, a SARS-CoV PLP2 corresponding to residues 1414-1858 of SARS-CoV nsp3 was expressed and purified from baculovirus-infected insect cells.

To prepare a nucleic acid encoding the SARS-CoV PLP2 polypeptide, a standard PCR reaction was conducted using a reverse-transcription product of SARS viral genome (SARS-TW1 strain) as a template. The following two oligonucleotides were used as primers: 5′-CGG GAT CCT GAA CTC TCT AAA TGA GCC GC-3′ (SEQ ID NO.: 3) and 5′-CGG AAT TCT TAC GAC ACA GGC TTG ATG GTT G-3′ (SEQ ID NO.: 4). The PCR products were digested with BamH I and EcoRI and ligated into a modified pBacPAK8/His2 vector (Chen et al., 2004, Protein Expr. Purif. 35, 142-146.) The resulting expression plasmid containing the PLP2 activity domain (from amino acids 1632 to 1847) and part of SARS-CoV unique domain (SUD) from pp1a of SARS-CoV with a 6×His tag at the N-terminus. The construct encoded the region from amino acids 1414 to 1858 with a calculated molecular weight of 51 kDa.

Protein was expressed in baculoviral infected insect cells (Berger et al., 1970, Philos. Trans. R. Soc. Lond. B. Biol. Sci. 257, 249-264.). Briefly, Sf9 cells were grown at 27° C. in Grace medium (Invotrogen) supplemented with 10% fetal bovine serum. Transfection of the cells with above-described expression vectors, and selection and amplification of the recombinant virus were carried out in the manner described in Chen et al., Protein Expr. Purif. 35, 142-146. To express the protein, Sf9 cells were infected with the recombinant virus at a multiplicity of infection of 3, and the cells were harvested 48 hours after infection by a standard method.

The insect cells were resuspended and sonicated in an equilibration buffer containing 20 mM Na₂HPO₄—NaH₂PO₄, 0.5 M NaCl, 20 mM imidazole and 7 mM β-mercaptoethanol, pH 8.2. The resulting cell lysate was cleared by centrifugation at 15000 g for 15 minutes, then loaded onto a His-Bind Fractogel affinity column. The column was washed with a buffer containing 20 mM imidazole then with a buffer containing 90 mM imidazole, before elution with an equilibration buffer containing 250 mM imidazole. The buffer of the eluate was changed to buffer A containing 20 mM Na₂HPO₄—NaH₂PO₄ and 7 mM β-mercaptoethanol, pH 6.2, using an Amicon Ultra-15 centrifugal filter tube, then loaded onto a Source 15S column. The Source 15S column was washed with buffer A, then eluted with a gradient of 0-1 M NaCl in buffer B containing 20 mM Na₂HPO₄—NaH₂PO₄ and 7 mM β-mercaptoethanol, pH 6.2. Western blot analysis was then carried out. It was found out that the purified protease ran approximately at the calculated size of 51 kDa in the SDS-PAGE gel and had a purity of over 95%. Approximately 0.2 mg of purified protein was obtained from one liter of insect cell culture.

Gel filtration experiment was carried out to determine the subunit composition of purified SARS-CoV PLP2 protease in the manner described in Sambrook et al., Molecular cloning: a laboratory manual. Cold Spring Harbor, N.Y.: Cold Spring Harbor Laboratory, 1989. After calibrating the column with proteins of known molecular mass and Stokes' radii, the purified 51 kDa SARS-CoV PLP2 protein was eluted at a position corresponding to 56.7 kDa protein with a Stokes' radius of 3.3 nm. The result indicates that the purified SARS-CoV PLP2 protease exists as monomer in solution.

The amino acid sequence of this 51 kDa protein contained several putative N- or O-glycosylation sites. Thus, whether the SARS-CoV PLP2 protein is glycosylated was investigated. DIG glycan differentiation kit (Roche), was used to detect several N- and O-link forms of glycosylation. The following glycosylation forms, including mannose, O-linked disaccharide galactose B (1-3) N-acetylgalactosamine, N-linked galactose-β (1-4)-N-acetylglucosamine, and O-linked N-acetylglucosamine, were not detected. The results indicate that that this purified SARS-CoV PLP2 protein is not glycosylated.

### EXAMPLE 2

A highly sensitive and quantitative method was developed to analyze the enzyme activity of SARS-CoV PLP2 in a system constituted of only purified enzyme and its substrate.

As mentioned above, SARS-CoV pp1a has three predicted SARS-CoV PLP2 sites, Gly180-Gly181, Gly818-Ala819, and Gly2740-Lys2741. However, the exact cleavage sites had not been confirmed yet. To confirm them, 12-mer oligopeptides encompassing these predicted cleavage sites were synthesized and used as SARS-CoV PLP2 substrates. These oligopeptides are RELNGGAVTRYV (SEQ ID NO.: 29), FRLKGGAPIKGV (SEQ ID NO: 8), and ISLKGGKIVSTC (SEQ ID NO.: 28), containing the Gly180-Gly181, Gly818-Ala819, and Gly2740-Lys2741 cleavage sites, respectively.

The cleavage reaction included peptide substrates and purified SARS-CoV PLP2 enzyme at concentrations of 1 mM and 1 μM, respectively, in a buffer containing 20 mM Na₂HPO₄—NaH₂PO₄ and 7 mM β-mercaptoethanol at pH 6.8. The reaction was carried out at 37° C. for either 2 or 12 hours followed by addition of 50 μl of 0.1% trifluoroacetic acid to stop the reaction. HPLC (RP-HPLC) RP-HPLC, Matrix-Assisted Laser Desorption/Ionization Time-of-Flight Mass Spectrometry (MALDI-TOF MS) and Liquid Chromatography Tandem Mass Spectrometry (LC-MS/MS) were then carried out to separate and quantify the cleavage products. More specifically, the product was analyzed on an Agilent 1100 HPLC system with a Zorbax C18 column (4.6×250 mm), using a 0-80% linear gradient of 90% acetonitrile with 0.1% trifluoroacetic acid. Cleaved products were collected and analyzed with a Voyager DE-STR Biospectrometry Workstation (PerSpective Biosystems, Framingham, Mass., USA). α-Cyano-4-hydroxycinnamic acid was used as matrix. For cleavage products of Gly180-Ala181 oligopeptides, the fraction was further analyzed on a LC-MS/MS system with a Zorbax column (2.1×250 mm) (LCQ DECA Xpplus, ThermoFinnigan, San Jose, Calif.), using the same gradient condition in RP-HPLC by replacing trifluoroacetic acid with acetic acid.

Substrate preference of SARS-CoV PLP2 was compared quantitatively and the exact cleavage site was determined. It was found that a small fraction (5%) of the Gly180-Gly181 substrate was cleaved after 12 hour digestion. Under the same condition, the cleavage of the Gly818-Ala819 and Gly2740-Lys2741 sites reached 100% and 29%, respectively. The percentage of the cleavage on the Gly818-Ala819 substrate reached 70% after 2 hours of digestion, while nearly none and only 7% were cleaved for the substrates containing Gly180-Gly181 and Gly2740-Lys2741 sites, respectively, indicating that the oligopeptide containing the Gly818-Ala819 site was the best substrate for SARS-CoV PLP2 in vitro. MALDI-TOF mass spectrometry also confirmed that the cleavage indeed occurred as predicted.

The above results demonstrate that SARS-CoV PLP2 cleaved the three predicted sites differentially with the preference toward the Gly818-Ala819 site. In addition, the result indicates that the observed activities of SARS-CoV PLP2 did not require the glycosylation of the protein.

The requirement for the substrate with respect to its length and composition was also studied. A series of Gly818-Ala819 site-containing oligopeptides ranging from 18-mer to 6-mer were synthesized and subjected to the above described assay. The results are shown in Table 1 below. The numbering of the P-P′ sites is based on Berger and Schechter (Berger et al., 1970, Philos. Trans. R. Soc. Lond. B. Biol. Sci. 257, 249-264).

TABLE 1
Optimal length for cleavage by SARS-CoV PLP2

| | Peptide Substrate | | | | | | | | | | | | | | | | | | Cleavage | %^{a} |
|---|---|---|---|---|---|---|---|---|---|---|---|---|---|---|---|---|---|---|---|---|
| Length | P9 | P8 | P7 | P6 | P5 | P4 | P3 | P2 | P1 | P1′ | P2′ | P3′ | P4′ | P5′ | P6′ | P7′ | P8′ | P9′ | 2 hours | 12 hours |
| 18^{a} | N | N | V | F | R | L | K | G | G | A | P | I | K | G | V | T | F | G | 60.5 ± 0.7 | 100 ± 0.0 |
| 16^{b} | | N | V | F | R | L | K | G | G | A | P | I | K | G | V | T | F | | 63.0 ± 1.4 | 100 ± 0.0 |
| 14 | | | V | F | R | L | K | G | G | A | P | I | K | G | VT | | | | 67.6 ± 9.0 | 100 ± 0.0 |
| 12 | | | | F | R | L | K | G | G | A | P | I | K | G | V | | | | 70.1 ± 7.4 | 100 ± 0.0 |
| 11 | | | V | F | R | L | K | G | G | A | P | I | K | | | | | | 52.9 ± 3.5 | 100 ± 0.0 |
| 10 | | | | F | R | L | K | G | G | A | P | I | K | | | | | | 49.9 ± 2.6 | 100 ± 0.0 |
| 10 | | | | | R | L | K | G | G | A | P | I | K | G | | | | | 14.8 ± 4.1 | 56.4 ± 5.6 |
| 9 | | | | F | R | L | K | G | G | A | P | I | | | | | | | 36.2 ± 6.4 | 85.1 ± 4.9 |
| 8 | | | | | | L | K | G | G | A | P | I | K | | | | | | 11.4 ± 4.3 | 44.7 ± 3.5 |
| 6 | | | | | | | K | G | G | A | P | I | | | | | | | n.d.^{c} | n.d |

^{a}Percentages of the cleavage is the average of three independent measurements using three different batches of enzyme. The enzymatic reaction was carried out for 2 or 12 hours before analysis and quantification by RP-HPLC.
^{b}Because of low solubility, 0.5 mM of the 18-mer and 16-mer oligopeptides were used. For the other substrates, a concentration of 1 mM was used.
^{c}Cleavage not detectable.

As shown in Table 1, oligopeptides ranging from 10-mer to 18-mer were optimal substrate for the cleavage, reaching 100% cleavage after 12 hours of digestion. 10-mer with six residues on the P sites and four on the P′ sites, i.e. FRLKGG-APIK, is optimal for the cleavage, reaching 50% and 100% after 2 hours and 12 hours of digestion, respectively. Same length oligopeptide with five each on P and P′ sites, RLKGG-APIKG, was less efficiently cleaved, with only 56% cleaved after 12 hours of incubation. Moreover, 9-mer with six residues at the P and three at P′ sites, FRLKGG-API, was a much better substrate than the 10-mer with five residues at the P sites. This 9-mer was 36% and 85% cleaved after 2 hour and 12 hour digestion, respectively. The results indicate that six residues on the P sites are critical for the optimal recognition and cleavage by the enzyme. Consistent with this, shorter oligopeptides (8-mer and 6-mer) without the P6 residue were poorly cleaved (11% and 45% cleaved after 2 hours and 12 hours of incubation, respectively), or not cleaved at all. Therefore, the P6 residue seems to contribute significantly to the substrate recognition and cleavage by SARS-CoV PLP2. 10-mer oligopeptide with six at the P sites seems to be the minimum requirement for the optimal cleavage by SARS-CoV PLP2 protease.

### EXAMPLE 3

Enzymatic properties of SARS-CoV PLP2 were studied. As described above, oligopeptide containing Gly818-Ala819 site is the best substrate for the SARS-CoV PLP2 enzyme in vitro. Thus, a polypeptide FRLKGGAPIKGV, which has the site, was used as substrate to study the kinetic properties of SARS-CoV PLP2.

The polypeptide was labeled with two fluorophores, o-Aminobenzoic acid (Abz) and N-ethylenediamine-2,4-dinitrophenyl amide (EDDNP) at its N- and C-termini, respectively. Abz and EDDNP were chosen as the fluorescent donor and quencher (Melo et al., 2001. Anal. Biochem. 293, 71-77) to produce an internally quenched fluorescent pair. When the polypeptide is intact, little fluorescent emits from it due to the fluorescence resonance energy transfer (FRET) between Abz (donor) and EDDNP (quencher). When the peptide substrate is cleaved, the FRET disappears and the fluorescence intensity increases. Enhanced fluorescence emission upon substrate cleavage was monitored at the excitation and emission wavelengths of 320 nm and 420 nm, respectively (Fluoroskan Ascent, ThermoLabsystems, Sweden). Fluorescence intensity was converted into amounts of hydrolyzed substrates according to a standard curve that had been generated based on the fluorescence measurements of the substrate with known concentrations after complete hydrolysis by SARS-CoV PLP2.

The kinetic constants were measured with this fluorogenic peptide substrate or fluorescent-free substrate in the manner described in Chien et al., 2004, J. Biol. Chem. 279, 52338-523345 and Polgar, 2002, Cell Mol Life Sci 59, 349-362. The concentrations of the enzyme and substrate used were 0.1 μM and 0.2-3 times of the Kₘ value (0.125 mM to 1 mM), respectively. The buffers used at different pH values were: a 20 mM acetate buffer (pH 5.25), a 20 mM phosphate buffer (pH 6-8), 20 mM Tris-HCl buffer (pH 8.65), or a 20 mM 3-cyclohexylamino-1-propanesulfonic acid (CAPS) buffer (pH 9.2-9.8). Each buffer contained 7 mM β-mercaptoethanol. The ionic strength change due to the pH adjustment was negligible with respect to total ionic strength. Peak area was calculated by integration. The initial rate was measured with less than 10% substrate depletion for the first 20 minutes to calculate the kinetic parameters using the Michaelis-Menten equation (Brocklehurst, 1996, Physical factors affecting enzyme activity. A. pH-dependent kinetics, Vol. A, BIOS Scientific Publisher Ltd, Oxford, UK and Dixon et al., 1979, Enzymes, 3rd ed., Academic Press). The results are shown in Table 2.

TABLE 2
Kinetic Parameters of SARS-CoV PLP2 at Different pHs^{a}

| pH | k_{cat} (min⁻¹) | Kₘ (μM) | K_{cat}/Kₘ (μM⁻¹min⁻¹) |
|---|---|---|---|
| 5.25 | 1.37 ± 0.21 | 21.45 ± 1.06 | 0.06 ± 0.01 |
| 6.05 | 11.90 ± 1.13 | 64.00 ± 0.28 | 0.17 ± 0.03 |
| 6.82 | 20.60 ± 2.40 | 65.45 ± 5.45 | 0.31 ± 0.01 |
| 7.42 | 19.75 ± 2.33 | 77.20 ± 2.12 | 0.27 ± 0.03 |
| 8.01 | 21.85 ± 1.77 | 71.95 ± 4.31 | 0.30 ± 0.02 |
| 8.65 | 22.00 ± 3.11 | 71.45 ± 7.99 | 0.30 ± 0.01 |
| 9.20 | 14.05 ± 0.50 | 68.05 ± 0.64 | 0.21 ± 0.02 |
| 9.80 | 5.99 ± 0.38 | 70.45 ± 1.49 | 0.08 ± 0.01 |

^{a}The results are the average of three independent measurements using three different batches of the enzyme.

It was found that the apparent k_{cat} and Kₘ values of SARS-CoV PLP2 at pH 7.4 were 20±2 min⁻¹ and 77±2 μM, respectively. The result indicates that the fluorescent substrate based method was much more sensitive than other methods (Kuo et al., 2004, Biochem. Biophys. Res. Commun. 318, 862-867).

Both k_{cat} and Kₘ values were determined at pH ranging from 5.25 to 9.8. The enzymatic activities for SARS-CoV PLP2 (k_{cat}/Kₘ values) were optimal in the pH range of 6.8 to 8.6. Lowering the pH to 5.25 affected both Kₘ and k_{cat} values, with more significant effect on the k_{cat}. The k_{cat} value decreased by almost 15-fold while the Kₘ value increased only by three folds. At pH 9.8, the k_{cat} decreased by three folds; no effect was found on the Kₘ value. The result demonstrates that the activity of SARS-CoV PLP2 is greatly modulated by pH.

The pH-log k_{cat} or pH-log (k_{cat}/Kₘ) profiles were fitted to the following equation for two protonation sites.

$$log k = log \frac{C}{1 + \frac{[ {H}^{+} ]}{{K}_{a 1}} + \frac{{K}_{a 2}}{[ {H}^{+} ]}}$$

in which k is the observed kinetic constant (k_{cat} or k_{cat}/Kₘ) at various pH values; C is the pH-independent limiting k value; and Kₐ₁ and Kₐ₂ are the macroscopic dissociation constants for the molecular groups responsible for the two ionization steps. All computer-fitted work was performed with the Sigma Plot 5.0 program (Jandel, San Rafael, Calif., USA). The information would reveal whether protonation of any ionic pairs affects the catalytic or the substrate binding efficiency.

It was found that both catalytic constant (k_{cat}) and specificity constant (k_{cat}/Kₘ) of the enzyme showed bell-shaped activity versus pH dependence, consistent with the existence of two macroscopic molecular ionizable groups for maximal catalysis. Apparent pKa values for these ionizable residues were obtained with an excellent fit in both cases.

From the pH-Log kcat plot, a constant C value of 25.4±3.43 min⁻¹, equivalent for the pH-independent k_{cat} value, was obtained. This was the true k_{cat} value and higher than the apparent value (20±2 min⁻¹) determined in the regular assay at fixed pH value of 7.4, as described above. From the pH-Log k_{cat} plot, two macroscopic molecular pKa values of 6.36±0.12 and 9.30±0.15 were obtained, most likely attributable to the dyad residues Cys1651 and His1812 of SARS-CoV PLP2. These results indicate that these two residues form the ion pair were involved in the catalytic step of the enzyme catalysis, and that the enzymatic activity was modulated electrostatically to be catalytically competent. The results were consistent with the characteristics of the papain-like cysteine protease (Pinitglang et al., 1997, Biochemistry 36, 9968-9982 and Storer et al., 1994, Methods Enzymol. 244, 486-500).

From the pH-Log (k_{cat}/Kₘ) plot, a constant C value of 0.31±0.02 min⁻¹μM⁻¹, equivalent for the pH-independent k_{cat}/Kₘ value, was obtained. This was the true k_{cat}/Kₘ value and the same as that determined in the regular assay at fixed pH value of 7.4, as described above.

In sum, k_{cat} and k_{cat}/Kₘ values were determined to be 25.4±3.4 min⁻¹ and 0.31±0.02 min⁻¹ μM⁻¹, respectively. Accordingly, the true Kₘ value for the 12-mer peptide substrate was corrected to 82 μM. Two apparent pKa values of 5.89±0.07 and 9.40±0.08, respectively, were obtained from the pH-Log (k_{cat}/Kₘ) plot. The former must be deprotonated and the latter protonated to reach an optimal substrate-binding mode. The above results suggest that the catalytic activity of SARS-CoV PLP2 is sensitive to pH, reflecting the stability of the thiolate-imidazolium ion pair at different pH values.

The stability of the protease at different temperatures was also determined by incubating the enzyme for 30 minutes at various temperatures before determining its initial rate at 37° C. The activity of the protease was significantly decreased after incubation at temperature higher than 37° C., indicating that the SARS-CoV PLP2 is inherently thermo-labile. This is consistent with the observation that SARS-CoV cannot tolerate high temperature.

### EXAMPLE 4

The substrate selectivity of several other coronaviral PLP proteases were studied by co-transfection study of the plasmid encoding the substrate with the substitution along with the PLPs, followed by immuno-precipitation and Western blot analysis. From such assays, it was not clear whether the cleavage was done solely by PLP protease or assisted by other cellular factors.

In this example, Reverse-phase HPLC (RP-HPLC) was used to study the substrate selectivity of SARS-CoV PLP2 followed by MALDI-TOF to confirm the cleavage sites. Previous studies on other coronaviral PLPs had shown that up to P6 site was important for the cleavage, while P′ sites were not as critical except the P1′ site. Therefore, this example focused on the P6 to P1 as well as P1′ sites.

A 12-mer substrate FRLKGGAPIKGV (SEQ ID NO: 8; P6-P1′ underlined), which contains the Gly818-Ala819 site, was used. Also used were series of peptides, each of which had a substitution at one of P6-P1′ (Table 3). Each peptide was incubated with purified SARS-CoV PLP2 before HPLC analysis. The results are shown in Table 3.

TABLE 3
Substrate Specificity of SARS-CoV PLP2

| | Peptide Substrateₐ | | | | | | | Cleavage %_{b} | |
|---|---|---|---|---|---|---|---|---|---|
| Source | P6 | P5 | P4 | P3 | P2 | P1 | P1′ | 2 h | 12 h |
| Gly818- | F | R | L | K | G | G | A | 70.1 ± 7.4 | 100 ± 0.0 |
| A1a819 | | | | | | | | | |
| A819G | • | • | • | • | • | • | G | 65.3 ± 10.2 | 100 ± 0.0 |
| A819N | • | • | • | • | • | • | N | 62.5 ± 12.2 | 100 ± 0.0 |
| G818A | • | • | • | • | • | A | • | n.d^{c} | n.d. |
| G817A | • | • | • | • | A | • | • | 16.1 ± 3.3 | 45.6 ± 6.6 |
| G816A | • | • | • | A | • | • | • | 71.2 ± 9.5 | 100 ± 0.0 |
| G816Q | • | • | • | Q | • | • | • | 58.3 ± 8.2 | 100 ± 0.0 |
| L815A | • | • | A | • | • | • | • | n.d. | n.d. |
| L815K | • | • | K | • | • | • | • | n.d. | n.d |
| R814A | • | A | • | • | • | • | • | 32.1 ± 6.3 | 82.1 ± 2.9 |
| R814S | • | S | • | • | • | • | • | 33.1 ± 4.9 | 85.2 ± 1.5 |
| F813A | A | • | • | • | • | • | • | 16.9 ± 2.4 | 56.5 ± 4.9 |
| F813V | V | • | • | • | • | • | • | 20.1 ± 5.2 | 64.4 ± 7.3 |

ₐThe substrates used in the study were all 12-mers. Only the P6 to P1′ residues are shown. Substitutions were as indicated. The other residues (not shown) are the same as in FRLKGGAPIKGV.
_{b}Percentage cleavage is the average of three independent measurements using three different batches of enzyme. The enzymatic reaction was carried out for 2 h or 12 h before analysis and quantification by HPLC.
^{c}Cleavage is not detectable.

As shown in Table 3, substitution at either P1 (Gly) or P4 (Leu) site (G818A, L815A or L815K) was not tolerated at all, with no cleavage even after 12 hours of digestion. The result indicates that there was a stringent requirement on these two sites for SARS-CoV PLP2. This is different from MHV PLP2, whose P4 (Leu) site is tolerant of change to Ala. Significant decrease of the cleavage, from 70% to around 20% after 2 hours, or from 100% to around 50% after 12 hours of incubation, were observed when P2 (Gly) or P6 (Phe) site was substituted (G817A, F813A or F813V). The results indicate that both P2 (Gly) and P6 (Phe) are important for the recognition and the cleavage by SARS-CoV PLP2. The substitution of P5 (Arg) with either Ala or Ser (R814A or R814S) resulted in moderately less cleavage, while substitution of P3 (Lys) with either Ala or Gln (K816A and K816Q) did not affect the cleavage at all. Furthermore, the SARS-CoV PLP2 was not selective on the P1′ site, since substitution of the P1′ (Ala) site with either Gly or Asn (A819G and A819N) did not affect the cleavage at all. In sum, P1, P2, P4 and P6 sites were important for optimal substrate recognition and cleavage by SARS-CoV PLP2. The result is consistent with the observation that the minimum optimal substrate cleavage requires the presence of P6 site.

### EXAMPLE 5

SARS-CoV PLP2 was studied for its ability to cleave proteins of MHV and BCoV. Listed in Table 4A are MHV or BCoV substrates (Bonilla et al., 1997, J. Virol. 71, 900-909; Herold et al., 1998, J. Virol. 72, 910-918; Ziebuhr et al., 2001, J. Biol. Chem. 276, 33220-33232; Lim et al., 1998, Adv. Exp. Med. Biol. 440, 173-184; Lim et al, 2000, J. Virol. 74, 1674-1685; Kanjanahaluethai et al., 2003, J. Virol. 77, 7376-7382; Dong et al., 1994, Virology 204, 541-549; Hughes et al., 1995, Adv. Exp. Med. Biol. 380, 453-458; and Chouljenko et al., J. Gen. Virol. 82, 2927-2933).

TABLE 4
Substrates of Coronaviral PLP Peptide Substrates^{a}

| Virus | AA^{b} | P6 | P5 | P4 | P3 | P2 | P1 | P1′ | Protease^{c} |
|---|---|---|---|---|---|---|---|---|---|
| SARS-CoV | 175 | R | E | L | N | G | G | A | PLP2 |
| SARS-CoV | 813 | F | R | L | K | G | G | A | PLP2 |
| SARS-CoV | 2735 | I | S | L | K | G | G | K | PLP2 |
| MHV | 2832 | F | S | L | K | G | G | A | PLP2 |
| BCoV | 2745 | F | S | L | K | G | G | A | PLP2? |
| HCoV-229E | 892 | F | T | K | A | A | G | G | PLP1/2 |
| IBV | 668 | V | V | C | K | A | G | G | PLP2 |
| IBV | 2260 | V | E | K | K | A | G | G | PLP2 |
| MHV | 242 | L | K | G | Y | R | G | V | PLP1 |
| MHV | 827 | W | R | F | P | C | A | G | PLP1 |
| BCoV | 241 | I | R | G | Y | R | G | V | PLP2 ? |
| BCoV | 846 | W | R | V | P | C | A | G | PLP1 ? |
| HCoV-229E | 106 | G | K | R | G | G | G | N | PLP1 |
| HCoV-229E | 892 | F | T | K | A | A | G | G | PLP1/2 |

^{a}Related Genbank accession numbers are AY291451 (SARS-CoV-TW1), NC_001846 (MHV), NC_003045 (BCoV), NC_002645 (HCoV-229E) and NC_001451 (IBV), respectively. The horizontal line below indicates the separation of the coronaviral PLP2s from PLP1s.
^{b}The starting position of the P6 residue.
^{c}The question mark denotes unconfirmed information.

As shown in Table 4, among the cleavage sites by MHV and HCoV-229E PLP1s, the sequences are poorly conserved except that the P1 site is either Gly or Ala. Interestingly, a consensus motif for the recognition of the substrate site by coronaviral PLP2 proteases seems to emerge, which is quite different from those of PLP1. SARS-CoV and MHV PLP2s share the consensus motif F/I-R/S-L-K-G-G from the P6 to P1 sites, while HCoV-229E and IBV share the conservation of A-G-G at the corresponding P2-P1-P1′ sites with the rest of the sites divergent. Notably, one of the sites in BCoV predicted cleavable by its PLP protease has the sequence of F-S-L-K-G-G from P6 to P1 sites, matching the consensus sequence of F/1-R/S-L-K-G-G postulated in this study.

It was found that SARS-CoV PLP2 can indeed process, though to a slightly less extend, the substrates of MHV or BCoV. The results are summarized in Table 5 below.

TABLE 5
SARS-CoV PLP2 Cleaves the Substrates of MHV and BCoV.

| | | | | | | | | Cleavage | |
|---|---|---|---|---|---|---|---|---|---|
| | Peptide Substrate^{a} | | | | | | | %^{b} | |
| Source | P6 | P5 | P4 | P3 | P2 | P1 | P1′ | 2 hours | 12 hours |
| Gly180- | R | E | L | N | G | G | A | n.d.^{c} | 5 ± 1 |
| Ala181 | | | | | | | | | |
| Gly818- | F | R | L | K | G | G | A | 70 ± 7 | 100 |
| Ala819 | | | | | | | | | |
| Gly2740- | I | S | L | K | G | G | K | 7 ± 1 | 29 ± 4 |
| Lys2741 | | | | | | | | | |
| MHV | F | S | L | K | G | G | A | 21 ± 5 | 80 ± 2 |
| BCoV | F | S | L | K | G | G | A | 27 ± 4 | 85 ± 9 |
| HCoV-229E | F | T | K | A | A | G | G | n.d. | n.d. |
| IBV | V | E | K | K | A | G | G | n.d. | n.d. |

^{a}The substrates used in the study were all 12-mers. Only positions P6 to P1′ are shown. The related GenBank accession numbers are AY291451 (SARS-CoV-TW1), Q9PYA3 (MHV), Q66198 (BCoV), Q05002 (HCoV-229E), and P27920 (IBV).
^{b}percentage cleavage is the average of three independent measurements using three different batches of enzyme.
^{c}Cleavage not detectable.

As shown in Table 5, at least 80% of MHV and BCoV substrates were cleaved after 12 hours of incubation, as compared to 100% cleavage of the Gly818-Ala819 site-containing SARS-CoV substrates. These MHV and BCoV substrates were better in vitro compared to the other two sites of SARS-CoV: Gly180-Gly181 and Gly2740-Lys2741. After 2 hours of digestion, SARS-CoV PLP2 cleaved MHV and BCoV substrates at about 21% and 27%, respectively, while it cleaved Gly180-Gly181 and Gly2740-Lys2741-containing substrate at only 5% and 7%, respectively. Furthermore, SARS-CoV PLP2 did not cleave HCoV-229E and IBV substrates. The study reveals that there is marked difference on substrate selectivity between coronaviral PLP1 and PLP2 enzymes, despite the fact that they are all dyad active site-containing papain-like proteases. SARS-CoV PLP2 is a selective enzyme on its substrate and it shares substrate homology with MHV and BCoV. The results also suggest that the site in a BCoV substrates is cleaved by its PLP2 instead of PLP1.

### EXAMPLE 6

The above-described fluorogenic peptide was used to screen for inhibitor of SARS-CoV PLP2.

Zinc ion was identified by this method to inhibit SARS-CoV PLP2 activity potently with the IC₅₀ value of 1.3 μM. Zinc conjugates, including N-ethyl-N-phenyldithiocarbamic acid Zn, hydroxypridine-2-thione Zn, and Dibenzyl dithiocarbamic Zn, were also found effective in inhibiting SARS-CoV PLP2. The inhibition is specific since other divalent metals, such as Mg, Mn, Ca, Ni and Co, had no effects on the activity of SARS-CoV PLP2 at 10 μM. Cu and Hg ion at 10 μM weakly inhibited the activity of the PLP2 to 70%. Also found were Azathioprine, Thiram, Carmustine, Thimerosal, Merbromin, 2-amino-6-mercaptopurine, and 6-mercaptopurine.

A number of commercially available cysteine protease inhibitors were tested. They include E64 (0.1 mM), N-ethylmaleimide (1 mM), cystatin (10 μg/ml), leupeptin (0.1 mM), antipain (0.1 mM), and chymostatin (1 mM). It was found that, except N-ethylmaleimide, none of them was effective in inhibiting SARS-CoV PLP2. These results suggest that the catalytic dyad of SARS-CoV PLP2 is not readily accessible to most of these inhibitory compounds.

The screening method uses only nanomolar amounts of SARS-CoV PLP2 and is suitable for high throughput screening.

As mentioned above, the controlled proteolysis of viral polypeptides by viral proteases is essential for viral virulence and biogenesis. In the above examples, it is demonstrated that, despite of low sequence homology, SARS-CoV PLP2 is a “papain-like” enzyme with similar catalytic mechanism as that of papain. Catalytically, SARS-CoV PLP2 is different from cathepsins, a papain-like cysteine proteases that also belongs to the papain super family but has narrow acidic pH optima (Lewis et al., 1978, J. Biol. Chem. 253, 5080-5086; and Menard et al., 1991, Biochemistry 30, 5531-5538).

The above mentioned catalytic Cys and His dyad is ubiquitously present among coronaviral PLPs and papain. However, the residues around the Cys and His dyad are quite different. In coronaviral PLPs, the nucleophilic Cys is preceded by Asn and followed by a hydrophobic (Y, F, or W) residue, while there is a signature motif GSCWAFS at the Cys active site of papain; the dyad residue His is preceded by a Gly (or Cys and Ser for PLP1s of BCoV and MHV, respectively) and followed by several hydrophobic residues, while Asp precedes the active site His of papain. Acidic groups near the dyad might contribute electrostatically to the modulation of the catalytically competent dyad ion pair (Cys⁻His⁺) (Pinitglang et al., 1997, Biochemistry 36, 9968-9982).

Site-specific mutagenesis might offer additional information to identify groups responsible for catalysis or substrate binding. Nevertheless, the wide pH-activity profiles indicate that this ionized thiolate-imidazolium ion pair is relatively more stable than is its neutral form (Storer et al., 1994, Methods Enzymol. 244, 486-500.35). PLP2s from both MHV and SARS-CoV are not inhibited by most commonly used cysteine protease inhibitors (see above and Kanjanahaluethai et al., 2000, J. Virol. 74, 7911-7921). In contrast, PLP1 from MHV is sensitive to these inhibitors (Kanjanahaluethai et al., 2000, J. Virol. 74, 7911-7921). These data suggest that the substrate-binding sites or the environments of the catalytic dyad in coronaviral PLP2s might be different significantly from those in PLP1 s or other E64-sensitive cysteine proteases, raising a possibility that structurally PLP1s might differ from their PLP2 counterparts.

The results described herein demonstrate that SARS-CoV PLP2 by itself is active, independent of contributions from the cellular environment or other cellular factors. Mass spectrometry is a sensitive alternative to determine cleavage sites as compared with known methods (Ziebuhr et al., 2001, J. Biol. Chem. 276, 33220-33232; Lim et al., 1994, Virology 204, 541-549; and Piccone et al., 1995, J. Virol. 69, 4950-4956.14, 36, 46). HPLC coupled with MASS spectrometry as described above not only confirmed the cleavage sites by SARS-CoV PLP2, but also detected the enzymatic activity of SARS-CoV not observable in vivo as described in Hartcourt et al., 2004, J. Virol. 78, 13600-13612. In the study described herein, cleavage at the Gly2740-Lys2741 site was detected by RP-HPLC, even though it was not detected in an in vivo study with a slightly longer PLP2 (residues 1540-2204). Only when the C-terminal region was extended to include a hydrophobic domain (residues 1540-2425) did cleavage of this Gly2740-Lys2741 site occur in vivo.

The study described herein shows that the intrinsic activity of SARS-CoV PLP2 processes all three predicted sites with differential activity, and that the extent of cleavage differs from that observed in vivo study. Purified SARS-CoV PLP2 marginally cleaved the Gly180-Ala181 site in vitro, with the greatest activity observed at the Gly818-Ala819 site (Table 5). However, the Gly180-Ala181 site was readily cleaved in vivo, seemingly with the greatest efficiency, whereas the other two sites, Gly818-Ala819 and Gly2740-Lys2741, were less efficiently processed (Hartcourt et al., 2004, J. Virol. 78, 13600-13612). The different results from these in vitro and in vivo studies may be due to the fact that the length and boundary of PLP domains affect the efficiency of the cleavage on the substrates in vivo (Ziebuhr et al., 2001, J. Biol. Chem. 276, 33220-33232; and Teng et al., 1999, J. Virol. 73, 2658-2666). The region outside the activity domain and/or the cellular localization of SARS-CoV PLP2 contributes to its activity observed in vivo.

SARS-CoV PLP2 is capable of cleaving polypeptides from MHV and BCoV, but not those from HCoV-229E and IBV (Table 5). Accordingly, it is speculated that the site from BCoV, FSLKGGA, is cleaved by its PLP2 rather than by its PLP1 (Table 5). Based on the common properties shared by MHV and BCoV on substrate homology and cleavage, and insensitivity to E64, the study described herein supports that SARS-CoV diverges early in evolution from MHV and BCoV. Therefore, rather than classifying SARS-CoV as an independent group 4 as originally proposed, the data described herein provide evidences to classify SARS-CoV phylogenetically as group 2b relative to group 2a, which includes MHV and BCoV. The information provided in this study is important for SARS-CoV-related research and epidemiology, because it validates the use of MHV and BCoV as a model to characterize SARS-CoV genes/proteins and functions.

Different from MHV PLP2 and uniquely for SARS-CoV PLP2, P4 (Leu) is critical for substrate recognition and cleavage (Table 3). In addition, P6 site might contribute through anchoring of the substrate to the active site (Table 1). Thus, it is likely that peptidomimetic compound with a longer chain might work as inhibitors to inhibit the enzymatic activities.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. An isolated polypeptide comprising a sequence that includes X₋₅-X₋₄-X₋₃-X₋₂-X₋₁-X₊₁, wherein
X₋₅ is Arg, Ala, Ser, or Glu;
X₋₄ is Leu;
X₋₃ is Lys, Ala, Gln, or Asn;
X₋₂ is Gly or Ala;
X₋₁ is Gly; and
X₊₁ is Ala, Gly, Asn, Val, or Lys; and
the polypeptide, upon contact with SARS-CoV PLP2, murine hepatitis virus PLP, or bovine coronavirus PLP, is cleaved between residues X₋₁ and X₊.

2. The polypeptide of claim 1, wherein the sequence includes X₋₆-X₋₅-X₄-X₋₃-X₋₂-X₋₁-X₊₁-X₊₂-X₊₃, wherein
X₋₆ is Arg, Phe, or Ile;
X₊₂ is Pro, Val, or Ile; and
X₊₃ is Ile, Phe, Val, or Thr.

3. The polypeptide of claim 2, wherein the sequence includes R₋₆E₋₅L₋₄N₋₃G₂G₋₁A₊₁V₊₂T₊₃RYV (SEQ ID NO: 29).

4. The polypeptide of claim 2, wherein the sequence includes F₋₆R₋₅L₋₄K₋₃G₋₂G₋A₊₁P₊₂I₊₃KGV (SEQ ID NO: 8).

5. The polypeptide of claim 2, wherein the sequence includes I₋₆S₋₅L₋₄K₋₃G₋₂G₋₁K₊₁I₊₂V₊₃STC (SEQ ID NO: 28).

6. The polypeptide of claim 2, wherein the sequence is selected from the group consisting of SEQ ID NOs: 5-30.

7. The polypeptide of claim 1, wherein the polypeptide is at least 8 amino acids in length.

8. The polypeptide of claim 7, wherein the polypeptide is 12 to 2560 amino acids in length.

9. The polypeptide of claim 8, wherein the polypeptide is 12 to 2422 amino acids in length.

10. The polypeptide of claim 9, wherein the polypeptide is 12 to 818 amino acids in length.

11. A method of identifying an inhibitor of SARS-CoV PLP2, murine hepatitis virus (MHV) PLP, or bovine coronavirus (BCoV) PLP, comprising:
(a) mixing, in a first sample, a test compound, a polypeptide of claim 1, and an enzyme selected from the group consisting of SARS-CoV PLP2, MHV PLP, and BCoV PLP; and
(b) detecting a first level of cleavage of the polypeptide by the enzyme, wherein the test compound is determined to be an inhibitor of SARS-CoV PLP2, MHV PLP, or BCoV PLP if the first level is lower than a second level determined from a second sample in the same manner, except the second sample is free of the compound.

12. The method of claim 11, wherein the enzyme contains SEQ ID NO: 1 or a functional equivalent thereof.

13. The method of claim 11, wherein the polypeptide is labeled at its amino-terminus and carboxyl-terminus respectively by a first fluorophore and a second fluorophore, one of the first and second fluorophores being a donor fluorophore and the other being an acceptor fluorophore, so that, when the polypeptide is intact, the donor fluorophore and the acceptor fluorophore are in close proximity to allow fluorescence resonance energy transfer therebetween; and step (c) comprising monitoring fluorescent emission change of either the acceptor fluorophore or the donor fluorophore upon irradiation of the donor fluorophore with an excitation light, the change being a function of the cleavage of the polypeptide.

14. The method of claim 13, wherein the donor fluorophore and acceptor fluorophore are o-aminobenzoyl (Abz) and hromophore, 2,4 nitrophenyl (Dnp), respectively, or EDANS and dabcyl, respectively.

15. The method of claim 11, wherein step (b) comprising conducting mass spectrometry.

16. A method of identifying a compound for treating an infection with SARS-CoV, murine hepatitis virus, or bovine coronavirus, comprising:
mixing a test compound and a polypeptide of claim 1, and
detecting presence of a binding between the test compound and the polypeptide, wherein the test compound is determined to be a candidate compound for treating the infection if a binding exists between the compound and the polypeptide.

17. A method of treating an infection with SARS-CoV, murine hepatitis virus, or bovine coronavirus, comprising administering to a subject in need thereof an effective amount of a polypeptide of claim 1 or an inhibitor of SARS-CoV PLP2, murine hepatitis virus (MHV) PLP, or bovine coronavirus (BCoV) PLP.

18. The method of claim 17, wherein the inhibitor is selected from the group consisting of a zinc salt, Azathioprine, Thiram, Carmustine, Thimerosal, N-ethylmaleimide, and Merbromin.

19. The method of claim 18, wherein the salt is N-ethyl-N-phenyldithiocarbamic acid Zn, Hydroxypridine-2-thione Zn, Dibenzyl dithiocarbamic Zn, or ZnCl₂.

20. The method of claim 17, wherein the inhibitor is a compound of formula (I): 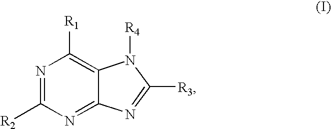
wherein
one of R₁, R₂, and R₃ is SR; and each of the others independently, is H, halo, C₁-C₁₅ alkyl, C₃-C₂₀ cycloalkyl, C₃-C₂₀ heterocycloalkyl, heteroaryl, aryl, SR, OR, or NRR′; and
R₄ is H, C₁-C₁₅ alkyl, C₃-C₂₀ cycloalkyl, C₃-C₂₀ heterocycloalkyl, heteroaryl, or aryl;
in which each R and R′, independently, is H, C₁-C₁₅ alkyl, C₃-C₂₀ cycloalkyl, C₃-C₂₀ heterocycloalkyl, heteroaryl, or aryl; or a salt thereof.

21. The method of claim 20, wherein the compound is 2-amino-6-mercaptopurine or 6-mercaptopurine.

22. An isolated nucleic acid comprising a sequence encoding the polypeptide of claim 1.

23. A vector comprising the nucleic acid of claim 22.

24. A host cell comprising the nucleic acid of claim 23.
